(19) United States
(12) Patent Application Publication
(10) Pub. No.: US 2026/0272702 A1
(43) Pub. Date: Sep. 17, 2026

(54) Pessary

(71) Applicant: Amara Therapeutics, Galway (IE)

(72) Inventors: Brendan Staunton, Dublin 18 (IE); Emma Carr, Galway (IE); Geoffrey Cundiff, North Vancouver, BC (CA)

(21) Appl. No.: 19/157,165

(22) PCT Filed: Feb. 16, 2024
(86) PCT No.: PCT/EP2024/053989

§ 371 Date: Aug. 15, 2025

(30) Foreign Application Priority Data
Feb. 16, 2023 (GB) 2302195.9

(51) Int. Cl. A61F 6/08

(52) U.S. Cl. CPC A61F 6/08

(57) ABSTRACT

A pessary (100) comprising an inflatable element (110) and an inflator (120). The inflatable element (110) is operable between a deflated state and an inflated state, wherein in the inflated state the inflatable element comprises an unobstructed external shape substantially similar to the anatomical shape of a human vagina. The inflator (120) is for operating the inflatable element (110) between the deflated state and the inflated state, and is configured to inflate the inflatable element to a pre-determined volume which is selected based on an optimal volume determined for a user of the pessary.

### TECHNICAL FIELD

The present invention relates to a pessary, and a device and method for fitting a pessary, and in particular but not exclusively to a pessary comprising an inflatable element.

### BACKGROUND

POP is a failure of pelvic floor support resulting in descent of one or more of the anterior, posterior, or apical pelvic floor compartments. POP occurs due to failure of the pelvic support structures including the levator ani muscle complex, cardinal and uterosacral ligaments, and endopelvic fascial connections. The anatomical failure of POP can be identified on physical exam but impacts patients through a variety of associated patient-reported symptoms.

POP impacts approximately 50% of all women. 3% to 6% of those women will have descent of prolapse beyond the hymen, which is well-correlated with the onset of patient symptoms. POP increases in prevalence and bother with increasing age, and changing demographics project that 9.2 million women will have POP by 2050. Risk factors for the development of POP include age, vaginal parity, obesity, connective tissue disorders, menopausal status, and chronic constipation.

Symptomatic POP is associated with urinary incontinence, depression, anxiety, sleep disturbance, sexual dysfunction, deteriorating physical function and diminished socialization. It has a substantial negative impact on the physical function and quality of life of women.

Treatment of POP is reserved for women with bothersome symptoms. Options for bothersome symptomatic POP include pelvic floor physical therapy, towards pelvic muscle strengthening, pessary fitting and use, and surgical intervention.

Pessaries, the mainstay of non-surgical treatment for POP, are orthotic devices placed in the vagina to provide temporary support. They have been used for centuries, for POP and other indications including stress urinary incontinence and cervical incompetence, with a wide variety of designs, and while there is significant evidence to support overall safety, complications including inadequate fit, discomfort, local tissue devascularization and erosion can limit use.

There are numerous pessaries available for treatment of POP. The majority fall into one of two broad categories: support pessaries, and space-filling pessaries. Support pessaries are typically characterized by the use of a spring mechanism that rests in the posterior fornix and against the posterior aspect of the symphysis pubis. With support pessaries, such as the ring pessary, vaginal support results from elevation of the superior vagina by the spring, which is supported by the symphysis pubis. Space-filling pessaries maintain their position in one of several ways, for example by creating suction between the pessary and vaginal walls (e.g., a cube pessary), by providing a diameter larger than the genital hiatus (e.g., a donut pessary), or by both mechanisms (e.g., a Gellhorn pessary).

Most clinicians begin by trying a support pessary, as they are typically easier to place and remove by the patient (for example, Gellhorn pessaries are much more difficult to remove than ring pessaries). Successful pessary use requires removal for cleaning at least every 3 to 6 months.

After an appropriate type of pessary is selected, the patient should be fitted with the largest size that can be comfortably worn, to prevent it from dislodging and falling out. An ideally fitted pessary should leave a patient unaware of its presence while in place. However, this is not routinely achievable for the majority of patients using currently available pessaries.

Most current guidelines advocate for and recommend pessaries as a first-line therapy before recommending surgery. Women who have failed a previous attempt at surgical management without relief often choose a pessary over additional surgery as well.

However, a significant proportion of women are unsuccessful at using a pessary. There are several different stages of pessary management where issues are experienced.

One barrier to pessary use is pessary avoidance, due to a lack of willingness on the part of patients to wear a pessary. A primary reason for pessary avoidance is patients' hesitance to manage their own pessary, which is often driven by the perceived difficulty experienced with both placing and removing a pessary.

Another barrier to pessary use is pessary fitting. Successful pessary fitting is often based on empiric experience, but even in the most experienced hands, 10-15% of patients cannot be successfully fit with a pessary. Moreover, accomplishing this level of successful fitting often requires trialling 2 to 3 different pessaries over multiple appointments. The most common reason for an unsuccessful fitting is an inability to hold the pessary in place (50%), followed by discomfort with the pessary (33%), unmasking of urinary incontinence (7%) and difficulty voiding (7%).

A third barrier to pessary use is pessary discontinuation. The success of pessary continuation (defined as continuing use of a pessary for longer than a month) ranges from 65% to 80%. Reasons given in the literature for discontinuation of pessary use include inability to retain the device, recurrent vaginal discharge or erosion, and unmasking of stress urinary incontinence or voiding dysfunction. Diabetes, stress urinary incontinence and lack of family support are significant risk factors for discontinuation of pessary use.

Unsuccessful fitting and discontinuation are both reflections of imperfect fit of a pessary. In the case of unsuccessful fit, the provider cannot find a pessary that remains in place without being uncomfortable. In the case of discontinuation of pessaries, this is usually driven by adverse events that occur due to mucosal injury that occurs due to rubbing associated with an imperfect fit.

The present invention has been devised with the foregoing in mind.

### SUMMARY

According to a first aspect, there is provided a pessary. The pessary may comprise an inflatable element. The inflatable element may be operable between a deflated state and an inflated state. In the inflated state the inflatable element may comprise an external shape substantially similar to the anatomical shape of a human vagina. In the inflated state, the inflatable element may comprise an unobstructed external shape substantially similar to the anatomical shape of a human vagina.

The term “unobstructed external shape” may refer to shape taken by the inflatable element in the inflated state without presence of external forces, for example, when inflated outside of a vagina into an empty space.

The current approach to pessary fitting utilizes a range of different pessaries that accommodate anatomical variation through different shapes and sizes that are not fundamentally based on actual female anatomy. They rely on one of several structural solutions to overcome anatomical failure that are not uniformly successful in all women with POP. This often results in an imperfect fit which can result in complications including vaginal discharge, pain, bleeding and abrasion or erosion of the vaginal mucosa that prevent continued use.

The pessary of the present invention may provide a space-filling pessary which in the deflated state may easily pass the vaginal opening to enable more comfortable fitting, but in the inflated state may become more rigid and fill the vaginal vault to provide support. As the vaginal vault has a large volume than the vaginal opening, in the inflated state the inflatable element may be less prone to coming out. The inflatable element may enable easy placement and removal of the pessary, encouraging continued use. The external shape of the inflatable element in the inflated state may substantially mirror the shape of the vaginal vault and accommodate the structures defining the vaginal shape to allow an improved fit. The inflatable element may also enable an improved and/or personalized fit by being inflatable to an appropriate (i.e., optimal) volume to ensure a successful fit. The improved fit which may be provided by the pessary of the present invention may therefore minimize adverse events that contribute to discontinuation.

The shape of the inflatable element in the inflated state may accommodate one or more anatomical structures that define the shape of the vagina, including the fascia endopelvinia, posterior fornix, uterosacral ligaments, arcus tendinous fascia pelvis, arcus tendinous fascia recotvaginalis, and muscles of the pubococcygeous and puborectalis slings. The inflatable element may comprise one or more dimensions corresponding to an anatomical structure of the vagina that are selected based on dimensions of the corresponding anatomical structure of a user.

The general shape of the vagina is defined by the underlying anatomical structures, and will not vary significantly between patients. The dimensions (for example, lengths) of specific structures and the resulting vaginal vault volume defined by the dimensions of those specific structures may vary between patients. Providing an inflatable element having an external shape substantially similar to the anatomical shape of the vagina in an inflated state, having at least one dimension corresponding to an anatomical structure of the vagina selected based on a dimension of the corresponding anatomical structure of a user, may enable an improved and/or personalized fit.

An anterior surface of the inflatable element may comprise a depression or recess. When inflated, the depression or recess may be configured to act as a suction cup. That may enable the pessary to provide additional support. An anterior surface of the inflatable element may refer to an anterior surface of the inflatable in use when correctly positioned within the vagina.

When in use in the inflated state, the inflatable element may exert greater pressure against the anterior, lateral, and apical portions of the user's vagina than the posterior portion of the user's vagina.

The inflatable element may comprise a non-inflating portion. The non-inflating portion may be, or form part of, a posterior portion of the inflatable element. The non-inflating portion may be configured, in use, to abut the posterior wall of the user's vagina.

The inflatable element may be made from or comprise silicone.

The pessary may comprise an inflator for operating the inflatable element between the deflated state and the inflated state. The inflator may be configured to both inflate and deflate the inflatable element.

The inflator may be manually operable. That may provide a simple, convenient way for a user to operate (for example, inflate and/or deflate) the inflatable element. That may allow easy placement, operation and removal of the pessary by the patient, limiting anxiety about pessary management and encouraging pessary continuation. The inflator may be or comprise a plunger mechanism.

The inflator may be configured to be releasably locked or fixed in position when the inflatable element is in the inflated state (for example, when the plunger mechanism is in a depressed position). That may enable the inflatable element to maintain the inflated state and reduce a risk of deflation without deliberate manual interaction from a user.

The inflator may be configured to inflate the inflatable element to a pre-determined volume. The pre-determined volume may be selected based on an optimal volume determined for a user of the pessary. That may enable the inflatable element to be inflated to an appropriate volume for the user, providing an improved fit and reducing or eliminating adverse events.

The inflator may be attached to the inflatable element such that the inflator is positioned at or adjacent the vaginal opening in use.

The inflatable element may be configured to at least partially receive the inflator. The inflatable element may comprise an elongated neck configured to at least partially receive the inflator. The inflatable element may be configured to receive the inflator such that in the inflated state the inflator is substantially housed or disposed within the inflatable element. That may improve comfort for a user and reduce a risk of accidental deflation of the inflatable element in use.

The inflator may be made from or comprise medical grade plastic.

According to a second aspect, there is provided a kit of parts for assembling a pessary. The kit of parts may comprise a plurality of inflatable elements. Each inflatable element may be operable between a deflate state and an inflated state. In the inflated state each inflatable element comprises an external shape substantially similar to the anatomical shape of a human vagina. In the inflated state, each inflatable element may comprise an unobstructed external shape substantially similar to the anatomical shape of a human vagina. A dimension of at least one part of the inflatable element corresponding to an anatomical structure of the vagina may be different for each of the inflatable elements. The kit of parts may also comprise a plurality of inflators for operating the inflatable elements between the deflated state and the inflated state. Each inflator may be configured to inflate the inflatable elements to a pre-determined volume. The pre-determined volume may be different for each of the inflators. The kit of parts may be used for assembling a pessary according to the first aspect.

According to a third aspect, there is provided a pessary fitting device. The pessary fitting device may comprise an inflatable element. The inflatable element may be operable between a deflated state and an inflated state. In the inflated state the inflatable element may comprise an external shape substantially similar to the anatomical shape of a human vagina. In the inflated state the inflatable element may comprise an unobstructed external shape substantially similar to the anatomical shape of a human vagina. The pessary fitting device may comprise at least one sensor configured to monitor inflation of the inflatable element as the inflatable element is inflated. The pessary fitting device may be used to fit a pessary according to the first aspect.

By monitoring inflation of the inflatable element during fitting, an improved fit for a pessary may be determined which may reduce adverse events that commonly lead to pessary discontinuation. Common adverse events such as vaginal discharge, bleeding, abrasion and erosion are the result of microabrasion and devascularization due to a pessary that is too tight.

The at least one sensor may be configured to provide data indicative of overinflation of the inflatable element as the inflatable element is inflated. Overinflation may relate to or be defined by an inflation volume that causes or is likely to cause devascularization.

The output of the at least one sensor may be configured to vary (for example, increases or decreases) as the inflation volume of the inflatable element varies (for example, increases or decreases). The at least one sensor may be configured to indicate overinflation if an output of the sensor exceeds or drops below a threshold value (for example, depending on the nature of the output of the at least one sensor).

The at least one sensor may comprise a sensor configured to determine blood flow in a vaginal wall of a user. The at least one sensor may be or comprise a photoplethysmography sensor.

The at least one sensor may comprise a pressure sensor.

The at least one sensor may be disposed in or on the inflatable element.

In use the at least one sensor may be positioned or located at or adjacent at least one of the anterior fornix and the posterior fornix. The anterior and posterior fornices are the most common locations for abrasions and erosions due to a pessary that is too tight. Positioning the at least one sensor at or adjacent one or both of the anterior and posterior fornices may enable better monitoring and determination of a pressure of the inflatable element that minimizes or avoids such abrasions and erosions.

The pessary fitting device may further comprise a processor. The processor may be configured to determine, from data obtained by the at least one sensor, an optimal inflation volume of the inflatable element for a user. The processor may be configured to determine an optimal inflation volume of the inflatable element that does not cause devascularization.

According to a fourth aspect, there is provided a method of using a pessary. The method may comprise inserting an inflatable element into a vagina of the user in a deflated state. The method may then comprise inflating the inflatable element to an inflated state. In the inflated state the inflatable element may comprise an external shape substantially similar to the anatomical shape of a human vagina. The method may comprise using the pessary of the first aspect.

The method may comprise deflating the inflatable element to the deflated state. The method may then comprise removing the inflatable element from the vagina of the user.

According to a fifth aspect, there is provided a method of fitting a pessary to a user. The method may comprise inserting an inflatable element into a vagina of the user in a deflated state. The method may then comprise inflating the inflatable element to an inflated state. In the inflated state the inflatable element may comprise an external shape substantially similar to the anatomical shape of a human vagina. The method may further comprise obtaining data from at least one sensor configured to monitor inflation of the inflatable element as the inflatable element is inflated. The at least one sensor may be configured to provide data indicative of overinflation of the inflatable element as the inflatable element is inflated. The method may comprise using the pessary fitting device of the second aspect.

The method may comprise determining, from data obtained by the at least one sensor, an optimal inflation volume of the inflatable element. The method may comprise determining an optimal inflation volume of the inflatable element that does not cause devascularization.

Features which are described in the context of separate aspects and embodiments of the present invention may be used together and/or be interchangeable wherever possible. Similarly, where features are described in the context of a single embodiment for brevity, those features may also be provided separately or in any sub-combination. Features described in connection with the apparatus of the first aspect may have corresponding features definable with respect to the apparatus of the second aspect, the kit of parts of the third aspect, the method of the fourth aspect or the method of the fifth aspect, and vice versa, and those embodiments are specifically envisaged.

### BRIEF DESCRIPTION OF THE DRAWINGS

The present invention will now be described, by way of example only, with reference to the accompanying drawings in which:

FIGS. 1A to 1E show a pessary in accordance with an embodiment of the invention, the pessary comprising an inflatable element shown in a deflated state;

FIGS. 2A to 2E show the pessary of FIGS. 1A to 1E with the inflatable element shown in an inflated state;

FIGS. 3A to 3E show a pessary fitting device in accordance with an embodiment of the invention, the pessary fitting device comprising the inflatable element of the pessary shown in FIGS. 1A to 1E and FIGS. 2A to 2E;

FIG. 4 shows a method of using the pessary shown in FIGS. 1A to 1E and FIGS. 2A to 2E in accordance with an embodiment of the invention; and

FIG. 5 shows a method of using the pessary fitting device shown in FIGS. 3A to 3E in accordance with an embodiment of the invention.

Like reference numbers and designations in the various drawings indicate like elements.

### DETAILED DESCRIPTION

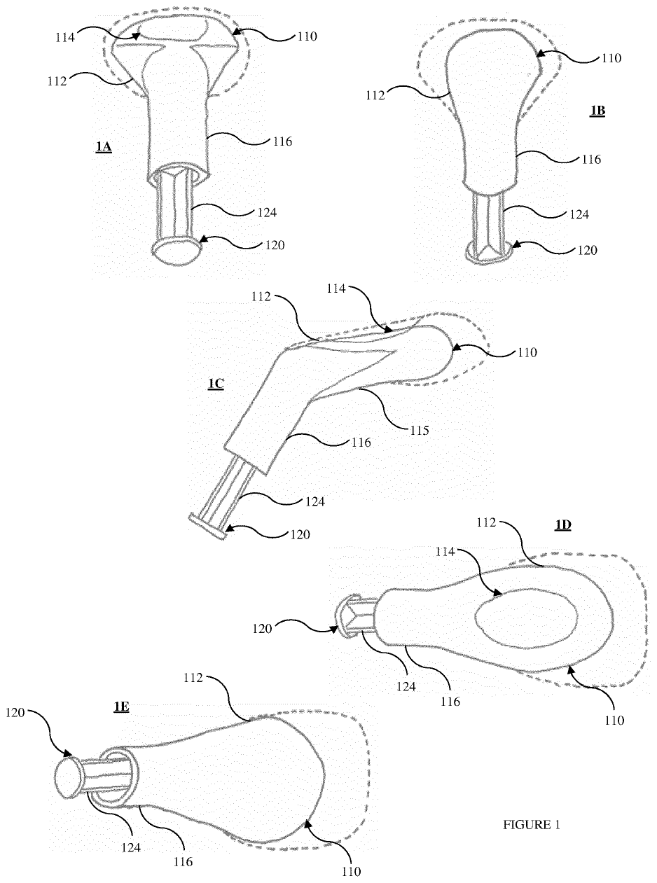

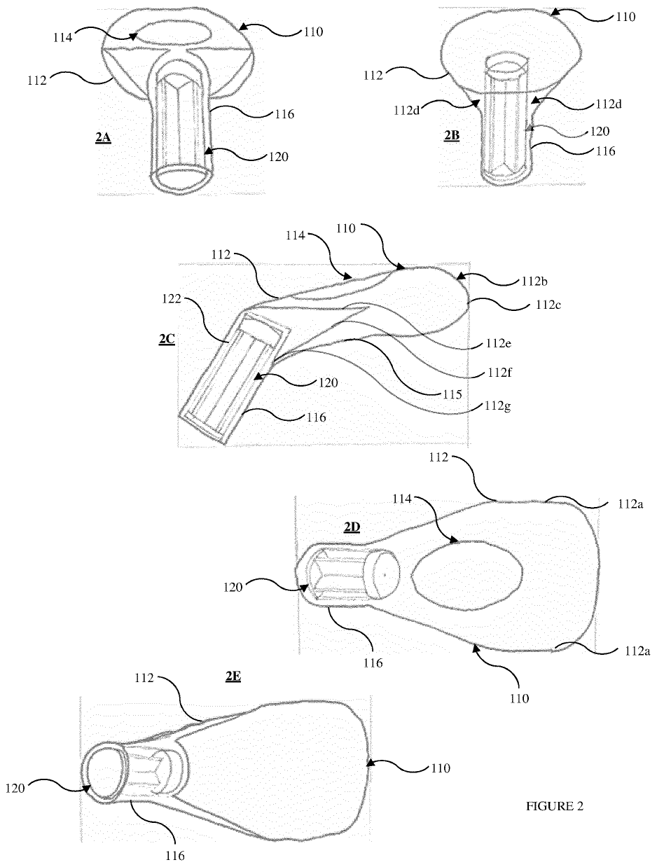

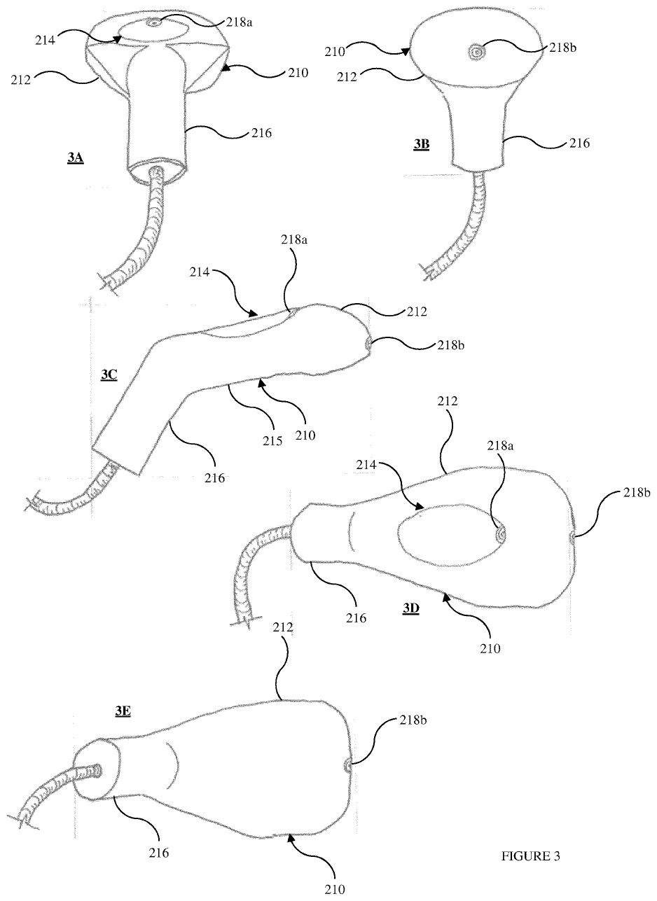

FIGS. 1A to 1E and 2A to 2E show a pessary 100 in accordance with an embodiment of the invention. The pessary 100 comprises an inflatable element or shell 110 operable between a deflated state and an inflated state. The inflatable element 110 is shown in the deflated state in FIGS. 1A to 1E, although the dashed lines illustrate an outline of the inflatable element 110 in the inflated state. The inflatable element 110 is shown in the inflated state in FIGS. 2A to 2E. The inflatable element 110 comprises a hollow structure configured to receive a fluid (for example, air or a liquid) to inflate the inflatable element 110 to the inflated state.

The inflatable element 110 comprises a main body 112. In the deflated state, the main body 112 of the inflatable element 110 is soft and has a small volume. That may enable the main body 112 to easily pass the vaginal opening for comfortable fitting. In the inflated state, the main body 112 of the inflatable element 110 is more rigid and has a larger volume. In particular, the main body 112 has a larger volume or cross-sectional area than the vaginal opening in the inflate state. That may prevent the main body 112 from inadvertently coming out of the vagina in use.

In at least the inflated state, the main body 112 comprises an external shape substantially similar to the anatomical shape of a human vagina. The general shape of the vagina is defined by the underlying anatomical structures, and does not vary significantly between patients. The structural elements that define the vaginal shape include the fascia endopelvinia, the posterior fornix, the uterosacral ligaments, the arcus tendinous fascia pelvis, the arcus tendinous fascia rectovaginalisa, the muscles of the pubococcygeous and the muscles of the puborectalis slings. In the inflated state, the external shape of the main body 112 is configured to act as a space-filling pessary that substantially mirrors the general shape of the vaginal vault, accommodating at least some of the anatomical structures that define the shape of the vagina.

As shown in FIGS. 2B, 2C and 2D, in the embodiment shown, different parts or regions of the main body 112 are configured to accommodate or correspond to different anatomical structures. First portions 112a of the main body 112 are configured to contact, accommodate or correspond to the fascia endopelvinia. A second portion 112b of the main body 112 is configured to contact, accommodate or correspond to the cervix or vaginal cuff following hysterectomy. A third portion 112c of the main body 112 is configured to contact, accommodate or correspond to the posterior fornix. Fourth portions 112d of the main body 112d are configured to contact, accommodate or correspond to the uterosacral ligaments. Fifth portions 112e of the main body 112 are configured to contact, accommodate or correspond to the arcus tendinous fascia pelvis. Sixth portions 112f of the main body 112 are configured to contact, accommodate or correspond to the arcus tendinous fascia rectovaginalisa. Seventh portions 112g of the main body 112 are configured to contact, accommodate or correspond to the muscles of the pubococcy geous and the muscles of the puborectalis slings. Only one of each of the fifth portions 112e, sixth portions 112f and seventh portions 112g is shown in FIG. 2C, although it will be appreciated a second respective portion is provided on the opposing side of the main body 112 (for example, as shown in FIG. 2B) The fifth 112e, sixth 112f and seventh 112g portions are formed by confluent edges or ridges provided in the main body 112 in the embodiment shown, although that is not essential.

The main body 112 is configured such that, when in use in the inflated state, pressure is applied by the main body 112 to the walls of the vagina differentially. The pressure exerted by the main body 112 to the walls of the vagina is non-uniform.

The main body 112 is configured such that, as it is inflated into the inflated state, it expands in the anterior, lateral, and apical portions of the vagina. The main body 112 is configured to inflate more in the anterior, lateral, and apical portions of the vagina than in the posterior region of the vagina. The main body 112 is configured such that there is no inflation (or less inflation than in other regions) in the posterior region of the vagina, where the main body 112 abuts the posterior wall of the vagina.

The main body 112 comprises portions 115 and 116 configured, in use, to abut the posterior wall of the vagina. Portions 115 and 116 are configured such that, as the main body 112 is inflated into the inflated state, they do not inflate. In some embodiments, the portions 115 and 116 of inflatable element 110 are connected to one or more struts or supports so as to prevent inflation of portions 115 and 116. As the user inserts the inflatable element 110 into their vagina, the portions 115 and 116 are brought into abutment with the posterior wall of the vagina, proximate the pelvic floor muscles. As the inflatable element 110 is inflated, portions 115 and 116 remain in abutment with the posterior wall of the vagina whilst the rest of the inflatable element 110 expands. This reduces the pressure exerted on the posterior wall of the vagina, and therefore reduces the pressure exerted upon the pelvic floor muscles of the user.

An outer surface of the main body 112 of the inflatable element 110 also comprises a depression or recess 114. The depression 114 is located on an anterior surface of the main body 112 (in use, when correctly positioned within the vagina). In the inflated state, the depression 114 is configured to act as a low-pressure suction cup to provide the pessary 100 with additional retention and support. As shown in FIG. 1C, the depression 114 is less perceptible in the deflated state. However, in the embodiment shown, the inflatable element 110 comprises one or more ribs or struts (not shown) disposed internally within the main body 112. The ribs or struts connect the anterior and posterior walls of the main body 112, resulting in the depression 114 becoming more emphasises as the inflatable element 110 is inflated. However, it will be appreciated the depression 114 may alternatively be provided in any suitable manner, for example by using a material for the main body 112 at or near the location of the depression 114 having a higher stiffness than the material used for the remainder of the main body 112. It will also be appreciated the inflatable element 110 may be provided without a depression or recess.

The inflatable element 110 also comprises a neck 116 connected to the main body 112. The neck 116 of the inflatable element 110 comprises an elongated structure configured to receive an inflator 120. The neck 116 provides a narrow opening for connecting to the inflator 120 in order to inflate or deflate the inflatable element. The elongated structure of the neck 116 also enables the inflator 120 to be received within the neck 116, and may improve ease of access and use for a patient by locating the inflator at or adjacent the vaginal opening in use. In the embodiment shown, the neck 116 is connected to the main body 112 such that the neck 116 is angled about 120 degrees relative to the main body 112. However, the neck 116 may be angled at any suitable angle relative to the main body 112, for example between about 100 degrees and about 140 degrees, or between about 110 degrees and 130 degrees. The neck 116 is connected to the main body 112 to enable the neck 116 to angle downwards in use (for example, away from the anterior surface of the main body 112) to reflect the location of the vaginal opening relative to the vaginal vault. That may further improve ease of access and comfort for a user. However, that is not essential, and the neck 116 may be connected to the main body 112 in any suitable manner.

In the embodiment shown, the inflator 120 comprises a plunger mechanism. The plunger mechanism comprises a cylinder 122 and a plunger 124 which moves along the longitudinal axis of the cylinder 122 to force fluid (for example, air or a liquid) into the inflatable element 110 or remove fluid from the inflatable element, thereby inflating or deflating the inflatable element 110. However, it will be appreciated any suitable inflator or inflation mechanism may alternatively be used. In the embodiment shown, the plunger mechanism is configured to inflate the inflatable element to a pre-determined volume (for example, when the plunger 124 is fully depressed within the cylinder 122). The pre-determined volume is based on an optimal volume determined for the user of the pessary 100. The same structure of the pessary 100 may therefore be inflated to different volumes to accommodate different vaginal vaults, thereby improving a personalised fit of the pessary 100 whilst using the same underlying structure. However, that is not essential, and the inflator 120 may be configured to provide any suitable volume to inflate the inflatable element 110 (for example, the inflator 120 may be or comprise a pump mechanism).

In the embodiment shown, the cylinder 122 is fully housed or disposed within the neck 116 of the inflatable element. In the deflated state, the plunger 124 extends from the neck 116 (as shown in FIGS. 1A to 1E). In the inflated state, when the plunger 124 is fully depressed within the cylinder 122, the plunger 124 is also housed or disposed within the neck 116, such that the plunger 124 no longer extends from the neck 116 (as shown in FIGS. 2A to 2E). However, that is not essential. It will be appreciated the cylinder 122 need not be fully located within the neck 116, and the plunger 124 may at least partially extend from the neck 116 even when fully depressed.

The cylinder 122 is held or secured within the neck 116, for example using an adhesive, although any suitable connection means may alternatively be used. In the embodiment shown, the cylinder 122 is secured within the neck 112 such that the inflator 120 and the inflatable element 110 are not removable or separable from one another after being connected. That may reduce a likelihood of the inflator 120 and inflatable element 110 becoming separated and reduce a likelihood of the pessary 100 coming out in use. Alternatively, the cylinder 122 may be held within the neck 116 such that the inflator 120 and the inflatable element 110 are separable or removable from one another, for example by a friction fit due to contact between an external surface of the cylinder 122 and an internal surface of the neck 124.

The plunger mechanism is configured such that when the plunger 124 is depressed within the cylinder 122 to inflate the inflatable element 110, the plunger 124 can be locked at full depression. The plunger 124 and the cylinder 122 may comprise complementary engaging structures (for example, a tongue and groove, a flange and recess, a sprung catch etc.) to allow the plunger 124 to be locked at full depression. For example, the plunger mechanism may comprise a sprung catch (not shown) that secures the plunger at full depression but can be released to allow subsequent withdrawal of the plunger 124 from the cylinder 122. The sprung catch may comprise a flexible protrusion or arm disposed on one of the plunger 124 and the cylinder 122, and a recess or aperture disposed on the other of the plunger 124 and the cylinder 122 configured to receive the arm or protrusion in the locked state. The arm or protrusion may be released (for example, by manual force on the arm or by sufficient axial or longitudinal pulling force applied to the plunger 124) to allow withdrawal of the plunger 124 from the cylinder 122. The complementary engaging features may be brought into engagement by placing the plunger 124 in the fully depressed state, or by rotating the plunger 124 within the cylinder while in the fully depressed state, For example, at full depression the plunger 124 may be rotatable within the cylinder 122 between a first rotational position in which the complementary engaging features are not engaged and a second rotational position in which the complementary structures are engaged. The complementary engaging structures may be located on an end of the plunger 124 and the cylinder adjacent an inlet or opening of the neck 116 when the plunger 124 is in the fully depressed state.

The inflatable element 110 is made from or comprises a silicone material in the embodiment shown. That may provide a medical grade material that can form a shell or balloon structure which can easily be inflated and deflated. However, any suitable alternative material may be used, for example a medical grade polymeric material capable of forming an inflatable structure such as nitrile rubber. The inflatable element 110 may be manufactured in a mould that defines the anatomical details of the external shape of the inflatable element 110 in the inflated state. The plunger mechanism of the inflator 120 is constructed of medical grade plastic in the embodiment shown.

The pessary 100 described above may provide a pessary which can be easily personalized to a specific patient's anatomy, which may improve a fit of the pessary and encourage pessary use and continuation.

As described above, the general shape of the vagina is defined by the underlying anatomical structures and does not vary significantly between patients. What varies is the lengths of the specific anatomical structures that define the shape, and the resulting vaginal vault volume that is defined by those lengths in combination.

In the embodiment shown, the pessary 100 comprises two main parts, the inflatable element 110 and the inflator 120. To improve a personalised fit, in an embodiment a kit of parts comprising a plurality of inflatable elements 110 and a plurality of inflators 120 (for example, plunger mechanisms) is provided. Each of the two parts 110, 120 may be selected from the respective plurality in order to provide a personalized fit of the pessary 100. Firstly, for the plurality of inflatable elements 110, a dimension of at least one part of the inflatable element 110 corresponding to an anatomical structure of the vagina is different for each of the inflatable elements 110. For example, 3 different sized inflatable elements 110 can be provided to accommodate different vaginal vaults. Secondly, for the plurality of inflators 120, each inflator 120 (for example, plunger mechanism) may be configured to inflate the inflatable elements 110 to a different pre-determined volume. For example, 10 different inflators 120 having different volumes can be provided (although any suitable number of different inflators having different volumes can be provided). A pessary 100 having a personalised fit may be constructed by selecting the most appropriate inflatable element 110 and the most appropriate inflator 120 for the user from the respective pluralities. That may enable the selected inflatable element 110 to be inflated by the inflator 120 to a pre-determined volume that substantially reflects an optimal volume for the user of the pessary 100.

By using a standard shape of the inflatable element 110 (based on the anatomical structure of the vagina) in different sizes and varying the inflation volume using a selected inflator 120, the pessary 100 may be constructed to accommodate substantially any normal vaginal morphology and provide an improved fit (when the inflatable element 110 is in the inflated state) whilst simultaneously enabling easy placement and removal of the pessary 100.

FIGS. 3A to 3E show an embodiment of a pessary fitting device 200 in accordance with an embodiment of the invention. The pessary fitting device comprises an inflatable element or shell 210 operable between a deflated state and an inflated state. In the embodiment shown, the inflatable element 210 is substantially similar to the inflatable element 110 described above with respect to FIGS. 1 and 2, with like elements referred to using like reference numerals. The inflatable element 210 is shown in the inflated state in FIGS. 3A to 3E.

The pessary fitting device 200 comprises sensors 218a, 218b. The sensors 218a, 218b are configured to monitor inflation of the inflatable element 210 as the inflatable element 210 is inflated. In the embodiment shown, the pessary fitting device 200 comprises two sensors 218a, 218b, although any suitable number of sensors may alternatively be used (one, three, four, five, six etc.).

The sensors 218a, 218b are located on the outer surface of the main body 212 of the inflatable element 210. A first sensor 218a is located or disposed on the inflatable element 210 such that in use (when correctly positioned within the vagina) the first sensor 218a is positioned at or adjacent the anterior fornix. In the embodiment shown, the first sensor 218a is located on an anterior surface of the main body 212 adjacent the depression 214, although it will be appreciated the first sensor 218a may be located in a substantially similar position even if the depression 214 is not present. Similarly, the second sensor 218b is located or disposed on the inflatable element 210 such that in use the second sensor 218b is positioned at or adjacent the posterior fornix. However, at least one sensor configured to monitor inflation of the inflatable element 210 may be positioned at any suitable location on the pessary fitting device 200.

In the embodiment shown, the sensors 218a, 218b each comprise a photoplethysmography (PPG) sensor. Each sensor 218a, 218b comprises a light source (for example, an LED) and a light sensor (for example, a photodiode) for measuring an amount of reflected light. With each cardiac cycle the heart pumps blood to the periphery. The pressure pulse that reaches the vaginal mucosa is damped, but still has sufficient intensity to distend the arteries and arterioles in the subcutaneous tissue. The change in volume caused by the pressure pulse can be detected by illuminating the vaginal mucosa with the light source and then measuring the intensity of light reflected back to the light sensor. An output from the light sensor shows each cardiac cycle as a peak. However, as the inflatable element 210 is inflated and the volume increases, the peak intensity is damped and reduces. If the volume of the inflated inflatable element 210 is too large (that is, the inflatable element 210 is overinflated), the resulting pressure can cause devascularization (obstruction or occlusion of the blood vessels leading to a loss of blood supply). That may cause the output of the PPH sensor to drop below a threshold value. Additionally, or alternatively, the at least one sensor 218 may be or comprise a pressure sensor. The pressure sensor 218 may provide a pressure measurement as the inflatable element 210 is inflated. If the inflatable element 210 is overinflated, the pressure measurement provided by the pressure sensor 218 may exceed a threshold pressure. The threshold pressure used to indicate overinflation of the inflatable element 210 may be pre-determined, for example a known pressure that is more likely than not to cause devascularization. The most common adverse events associated with pessary fitting and use (including vaginal discharge, bleeding, abrasion and erosion) are the result of microabrasion and devascularisation due to a pessary that is too tight.

By monitoring inflation in order to detect overinflation of the inflatable element 210 using the sensors 218a, 218b, a maximum volume in the inflatable element 210 that does not cause devascularization can be determined. In some embodiments, the pessary fitting device 200 comprises a processor configured to determine an optimal inflation volume of the inflatable element 210 based on data obtained by the sensors 218a, 218b. That may enable an optimal volume of the inflatable element 210 to be determined that provides a good fit (preventing the eventual pessary 100 from coming out) whilst minimizing the risk of adverse events due to pessary use (for example, due to devascularization resulting from an overinflated pessary 100 causing too tight a fit). The processor may be in wired or wireless communication with the sensors 218a, 218b, and may be located on or within the inflatable element 210 or may be located externally to the inflatable element 210 (for example, the processor may be part of a computing device external to the patient while the pessary fitting device 200 is in operation). In the embodiment shown, the inflatable element 210 is inflated via a conduit connected to a pump (not shown) in order to provide accurate control and monitoring over the inflation volume of the inflatable element 210. However, it will be appreciated the inflatable element 210 may be inflated in any suitable manner.

From the determined maximum or optimal inflation volume that prevents the pessary 100 from falling out without causing devascularization, a pessary 100 may be constructed as described above. In particular, an appropriate inflator 120 configured to inflate the inflatable element 110 to a pre-determined volume may be selected to be paired with an appropriately sized inflatable element. The pre-determined volume of the inflator 120 can be selected based on the determined maximum or optimal inflation volume, such that the pre-determined volume provided by the inflator 120 is substantially similar to the determined maximum or optimal inflation volume. In turn, the inflator 120 is configured to inflate the inflatable element 110 to an optimal volume to provide an improved, personalized fit whilst minimizing a risk of adverse events.

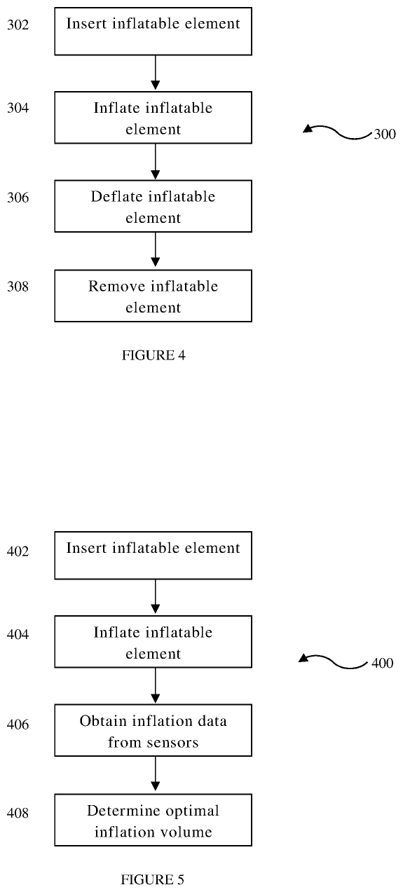

FIG. 4 shows a method 300 of using the pessary 100. Step 302 of the method 300 comprises inserting the inflatable element 110 into a vagina of the user in a deflated state. Step 304 of the method 300 comprises inflating the inflatable element 110 to an inflated state. In the inflated state the inflatable element 110 comprises an external shape substantially similar to the anatomical shape of a human vagina, as described above. Step 306 of the method comprises deflating the inflatable element to the deflated state. In the embodiment shown, steps 304 and 306 are performed by an inflator 120 comprising a plunger mechanism to inflate the inflatable element 110 to a pre-determined volume and subsequently deflate the inflatable element 110. However, it will be appreciated an alternative inflator 120 may be used to inflate and deflate the inflatable element, as described above. Step 308 of the method 300 comprises removing the inflatable element from the vagina of the user.

FIG. 5 shows a method 400 of using the pessary fitting device 200. Step 402 of the method 400 comprises inserting the inflatable element 210 into a vagina of the user in a deflated state. Step 404 of the method 400 comprises inflating the inflatable element 210 to an inflated state. The inflatable element 210 may be inflated by incrementally increasing a volume with each increment having a pre-determined volume, or by continuously increasing a volume of the inflatable element 210. In the inflated state the inflatable element 210 comprises an external shape substantially similar to the anatomical shape of a human vagina, as described above. Step 406 of the method 400 comprises obtaining data from at least one sensor (for example, sensors 218a, 218b located on the inflatable element 210) configured to monitor inflation of the inflatable element 210 as the inflatable element 210 is inflated. Step 408 of the method 400 comprises determining, from data obtained by the at least one sensor, an optimal inflation volume of the inflatable element 210. In the embodiment shown, step 408 comprises determining an optimal inflation volume of the inflatable element that does not cause devascularization, although that is not essential. If the inflatable element 210 is inflated by incrementally increasing a volume, the optimal volume may be the highest volume increment that does not cause devascularization.

From reading the present disclosure, other variations and modifications will be apparent to the skilled person. Such variations and modifications may involve equivalent and other features which are already known in the art of pessaries and/or pelvic floor dysfunction, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom.

For the sake of completeness, it is also stated that the term “comprising” does not exclude other elements or steps, the term “a” or “an” does not exclude a plurality, a single processor or other unit may fulfil the functions of several means recited in the claims and any reference signs in the claims shall not be construed as limiting the scope of the claims.

1. A pessary comprising:
an inflatable element operable between a deflated state and an inflated state, wherein in the inflated state the inflatable element comprises an unobstructed external shape substantially similar to the anatomical shape of a human vagina; and
an inflator for operating the inflatable element between the deflated state and the inflated state, wherein the inflator is configured to inflate the inflatable element to a pre-determined volume which is selected based on an optimal volume determined for a user of the pessary.

2. The pessary of claim 1, wherein an anterior surface of the inflatable element comprises a depression or recess.

3. The pessary of claim wherein the inflatable element is made from or comprises silicone.

4. The pessary of claim 1, wherein the inflator is manually operable.

5. The pessary of claim 4, wherein the inflator is or comprises a plunger mechanism.

6. The pessary of claim 1, wherein the inflatable element is configured to at least partially receive the inflator.

7. The pessary of claim 6, wherein the inflatable element comprises an elongated neck configured to at least partially receive the inflator.

8. The pessary of claim 1, wherein, when in use in the inflated state, the inflatable element exerts greater pressure against the anterior, lateral, and apical portions of the user's vagina than the posterior portion of the user's vagina.

9. The pessary of claim 1, wherein the inflatable element comprises a non-inflating portion configured, in use, to abut the posterior wall of the user's vagina.

10. The pessary of claim 1, wherein the inflator is made from or comprises medical grade plastic.

11. A pessary fitting device comprising:
an inflatable element operable between a deflated state and an inflated state, wherein in the inflated state the inflatable element comprises an unobstructed external shape substantially similar to an anatomical shape of a human vagina; and
wherein the pessary fitting device comprises at least one sensor configured to monitor inflation of the inflatable element as the inflatable element is inflated.

12. The pessary fitting device of claim 11, wherein the at least one sensor is configured to provide data indicative of overinflation of the inflatable element as the inflatable element is inflated.

13. The pessary fitting device of claim 12, wherein the at least one sensor is configured to indicate overinflation if:
a) an output of the sensor exceeds a threshold value; or
b) an output of the sensor drops below a threshold value.

14. The pessary fitting device of claim 11, wherein the at least one sensor comprises a sensor configured to determine blood flow in a vaginal wall of a user, and optionally wherein the at least one sensor comprises a photoplethysmography sensor.

15. The pessary fitting device of claim 11, wherein the at least one sensor comprises a pressure sensor.

16. The pessary fitting device of claim 11, wherein the at least one sensor is disposed in or on the inflatable element.

17. The pessary fitting device of claim 16, wherein in use the at least one sensor is located at or adjacent at least one of the anterior fornix and the posterior fornix.

18. The pessary fitting device of claim 11, further comprising a processor configured to determine, from data obtained by the at least one sensor, an optimal inflation volume of the inflatable element for a user, and optionally configured to determine an optimal inflation volume of the inflatable element that does not cause devascularization.
